(19) United States
(12) Patent Application Publication
(10) Pub. No.: US 2014/0163374 A1
(43) Pub. Date: Jun. 12, 2014

(54) ULTRASOUND DIAGNOSIS APPARATUS

(71) Applicants: TOSHIBA MEDICAL SYSTEMS CORPORATION, Tochigi (JP); KABUSHIKI KAISHA TOSHIBA, Tokyo (JP)

(72) Inventors: Yoichi OGASAWARA, Nasushiobara-shi (JP); Daizo OIKAWA, Otawara-shi (JP); Keisuke HASHIMOTO, Nasushiobara-shi (JP)

(73) Assignees: TOSHIBA MEDICAL SYSTEMS CORPORATION, Tochigi (JP); KABUSHIKI KAISHA TOSHIBA, Tokyo (JP)

(21) Appl. No.: 14/177,271

(22) Filed: Feb. 11, 2014

Related U.S. Application Data
(63) Continuation of application No. PCT/JP2012/074262, filed on Sep. 21, 2012.

(30) Foreign Application Priority Data
Sep. 22, 2011 (JP) 2011-207203
Sep. 21, 2012 (JP) 2012-208086

(51) Int. Cl. A61B 8/00

(52) U.S. Cl. CPC A61B 8/4236
USPC 600/443

(57) ABSTRACT

An ultrasound diagnosis apparatus according to an aspect includes an ultrasound probe and a processing apparatus. The ultrasound probe is configured so that a contact face thereof to be in contact with a subject for the purpose of adhering thereto is formed so as to have a shape that can be fitted to a projection part of the subject. The processing apparatus processes a reflected-wave signal of an ultrasound wave that is transmitted from the ultrasound probe attached to the subject toward the subject.

### CROSS-REFERENCE TO RELATED APPLICATIONS

This application is a continuation of PCT international application Ser. No. PCT/JP2012/074262 filed on Sep. 21, 2012 which designates the United States, incorporated herein by reference, and which claims the benefit of priority from Japanese Patent Application No. 2011-207203, filed on Sep. 22, 2011; and Japanese Patent Application No. 2012-208086, filed on Sep. 21, 2012, the entire contents of which are incorporated herein by reference.

### FIELD

Embodiments described herein relate generally to an ultrasound diagnosis apparatus.

### BACKGROUND

Conventionally, ultrasound diagnosis apparatuses have been used in today's medicine for performing a medical examination and making a diagnosis on various tissues in the body of an examined subject (hereinafter, “subject”) such as the heart, the liver, a kidney, a mammary gland, and the like, because ultrasound diagnosis apparatuses have advantages realized by simpler operability and non-invasiveness (i.e., no possibility of causing radiation exposures) over other medical image diagnosis apparatuses such as X-ray diagnosis apparatuses and X-ray computed tomography apparatuses. For example, an ultrasound diagnosis apparatus is configured to, when an ultrasound probe is pressed against a subject by an operator such as a medical doctor, generate an ultrasound image that is an image of a structure of a tissue inside the subject, by receiving a reflected-wave signal obtained when an ultrasound wave transmitted from the ultrasound probe is reflected by the tissue inside the subject. Consequently, the ultrasound diagnosis apparatus is capable of generating ultrasound images of mutually-different tissues in correspondence with the sites against which the ultrasound probe is pressed by the operator.

### BRIEF DESCRIPTION OF THE DRAWINGS

FIG. 1 is a drawing of an exemplary configuration of a diagnosis system according to a first embodiment;

FIG. 2 is a drawing for schematically illustrating an exterior appearance of a diagnosis apparatus according to the first embodiment;

FIG. 3 is a drawing for schematically illustrating an exterior appearance of an ultrasound probe according to the first embodiment being attached to a subject P;

FIG. 4 is an enlarged view of an exterior appearance of the ultrasound probe in the direction of an arrow A1 in FIG. 3;

FIG. 5 is an enlarged view of an exterior appearance of the ultrasound probe in the direction of an arrow A2 in FIG. 3;

FIG. 6 is a cross-sectional view of the ultrasound probe at a line I1-I1 in FIG. 5;

FIG. 7 is a schematic drawing of a state of the ultrasound probe fixed in an intercostal region;

FIG. 8 is an enlarged view of an exterior appearance of a two-dimensional ultrasound probe according to the first embodiment;

FIG. 9 is a diagram of an exemplary configuration of an apparatus main body according to the first embodiment;

FIG. 10 is a flowchart of a processing procedure performed by the diagnosis apparatus according to the first embodiment;

FIG. 11 is an enlarged view of an exterior appearance of an ultrasound probe according to a first modification example;

FIG. 12 is a cross-sectional view of the ultrasound probe at a line I2-I2 in FIG. 11;

FIG. 13 is a drawing of an exterior appearance of the ultrasound probe according to the first modification example being fixed onto a subject;

FIG. 14 is an enlarged view of an exterior appearance of an ultrasound probe according to a second modification example;

FIG. 15 is a cross-sectional view of the ultrasound probe at a line I3-I3 in FIG. 14;

FIG. 16 is a cross-sectional view of an ultrasound probe according to a third modification example; and

FIG. 17 is a drawing of an example of an exercise-stress cardiac echo test performed by a diagnosis apparatus according to an exemplary embodiment.

### DETAILED DESCRIPTION

An ultrasound diagnosis apparatus according to an aspect includes an ultrasound probe and a processing apparatus. The ultrasound probe is configured so that a contact face thereof to be in contact with a subject for the purpose of adhering thereto is formed so as to have a shape that can be fitted to a projection part of the subject. The processing apparatus processes a reflected-wave signal of an ultrasound wave that is transmitted from the ultrasound probe attached to the subject toward the subject.

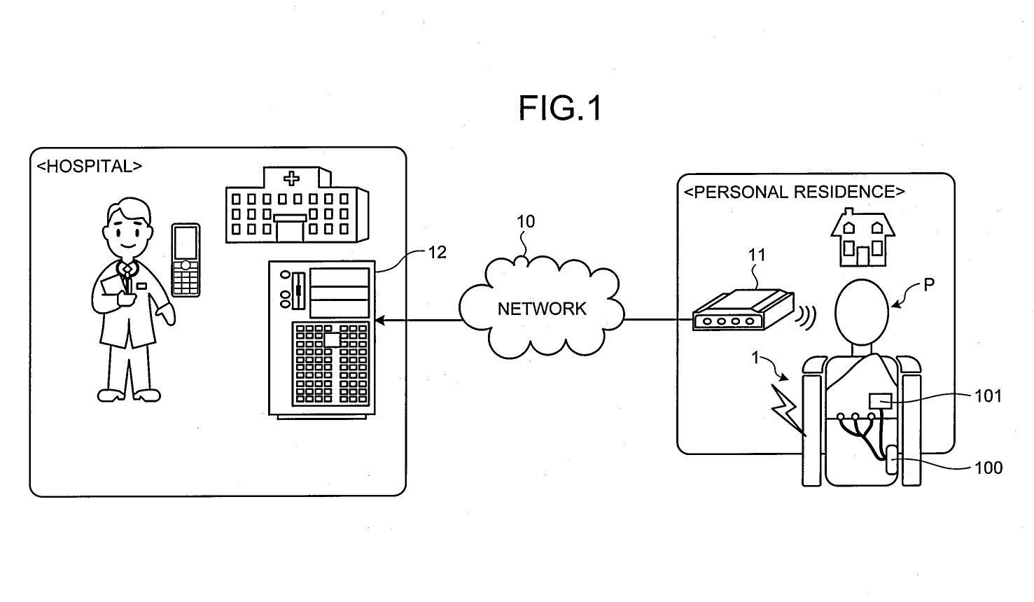

First, a diagnosis system according to a first embodiment will be explained, with reference to FIG. 1. FIG. 1 is a drawing of an exemplary configuration of the diagnosis system according to the first embodiment. As shown in FIG. 1, the diagnosis system according to the first embodiment in an exemplary mode includes: a diagnosis apparatus 1 attached to a subject P who is in a personal residence; an access point 11 such as a wireless router installed in the personal residence of the subject P; and a server apparatus 12 installed in a hospital. The access point 11 and the server apparatus 12 are capable of communicating with each other via a network 10. For example, the access point 11 and the server apparatus 12 communicate with each other via a secure circuit such as a Virtual Private Network (VPN).

The diagnosis apparatus 1 is a portable ultrasound diagnosis apparatus that is carried by the subject P and is integrally provided with a Holter electrocardiography device. The diagnosis apparatus 1 is configured to wirelessly communicate with the access point 11. More specifically, the diagnosis apparatus 1 includes an apparatus main body 100 and an ultrasound probe 101 that is thin and can be fixed onto the subject P. The apparatus main body 100 is configured to regularly record an electrocardiogram (ECG) while the subject P is leading a daily life and to generate ultrasound images from reflected-wave signals of ultrasound waves transmitted from the ultrasound probe 101 to the subject P. Further, the apparatus main body 100 transmits the ECGs and the ultrasound images to the server apparatus 12 via the network 10, by regularly transmitting the ECGs and the ultrasound images to the access point 11.

The server apparatus 12 stores therein, for each examined subject (i.e., subject), personal information regarding the subject and various types of medical data such as ECGs and ultrasound images obtained from the subject. The server apparatus 12 according to the first embodiment is configured to accumulate therein the ECGs and the ultrasound images that are obtained from the subject P while the subject P is leading a daily life, by receiving the ECGs and the ultrasound images that are regularly transmitted from the apparatus main body 100. With this arrangement, a medical doctor or the like who is in the hospital is able to view the ECGs and the ultrasound images obtained from the subject P who is in the personal residence, by accessing the server apparatus 12 via a portable terminal or a personal computer.

In this situation, the apparatus main body 100 according to the first embodiment is configured to analyze the ECGs regularly acquired from the subject P. Further, when having detected an abnormality in the subject P as a result of analyzing the ECGs, the apparatus main body 100 generates an ultrasound image of the subject P by causing the ultrasound probe 101 to transmit an ultrasound wave. In other words, when there is a possibility that the subject P may have an abnormality as a result of the analysis using the ECGs, the apparatus main body 100 generates the ultrasound image by immediately scanning the subject P by using the ultrasound probe 101. Further, every time an ultrasound image is generated, the apparatus main body 100 transmits the ultrasound image to the server apparatus 12, together with the ECG corresponding to the time when the abnormality was detected. As a result, the diagnosis apparatus 1 according to the first embodiment makes it possible to examine the subject P while using not only the ECGs but also the ultrasound images, when there is a possibility that subject P may have an abnormality. Generally speaking, because ECGs are obtained from small electric signals flowing in the subject P, waveforms of the ECGs may change due to changes in the posture of the subject P, and waveforms of the ECGs may have noise due to body movements of the subject. However, the diagnosis apparatus 1 according to the first embodiment makes it possible to examine the subject P in an integral manner while using the ECGs and the ultrasound images. Thus, even if the waveforms of the ECGs are disturbed by the posture changes and/or the body movements of the subject P, it is possible to improve the level of precision of the diagnoses made by the medical doctor.

Next, the diagnosis apparatus 1 described above will be explained further in detail. In the following sections, the ultrasound probe 101 that makes it possible to examine the subject P in an integral manner will be explained first. Secondly, a configuration and a processing procedure of the apparatus main body 100 will be explained. In the first embodiment, an example will be explained in which the diagnosis apparatus 1 is configured to regularly record an ECG of the subject P and also to generate, when an abnormality has been detected, an ultrasound image of the chest (e.g., the heart) of the subject P. It should be noted, however, that it is acceptable to configure the diagnosis apparatus 1 so as to generate ultrasound images of a site (e.g., the abdomen) other than the chest.

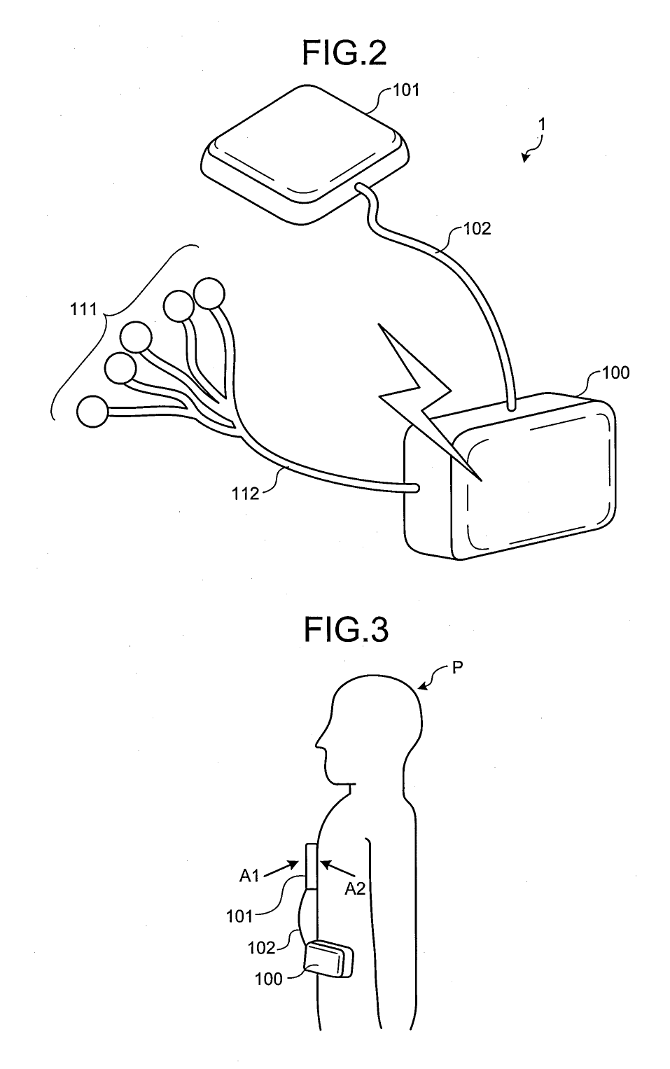

FIG. 2 is a drawing for schematically illustrating an exterior appearance of the diagnosis apparatus 1 according to the first embodiment. As shown in FIG. 2, the diagnosis apparatus 1 according to the first embodiment includes the apparatus main body 100, the ultrasound probe 101, and Holter electrocardiograph (ECG) probes 111.

The apparatus main body 100 and the ultrasound probe 101 are connected to each other by a cable 102 so as to allow electric communication therebetween, whereas the apparatus main body 100 and the Holter ECG probes 111 are connected to each other by a cable 112 so as to allow electric communication therebetween. The cable 102 and the cable 112 are bendable members and are configured with, for example, metal wires each covered with an electrically-insulating material such as rubber.

The Holter ECG probes 111 are fixed onto the body surface of the subject P by adhesive pads or the like and are configured to obtain ECG data by detecting small electric signals from the inside of the subject P. The ultrasound probe 101 is configured so that a contact face thereof to be in contact with the subject P for the purpose of adhering thereto is formed so as to have a shape that can be fitted to a projection part (e.g., a rib) of the subject P. The ultrasound probe 101 transmits an ultrasound wave to the subject P and receives a reflected-wave signal obtained when the ultrasound wave is reflected on the inside of the subject P. The apparatus main body 100 is a processing apparatus that processes the reflected-wave signal of the ultrasound wave that is transmitted from the ultrasound probe 101 attached to the subject P toward the subject P. More specifically, the apparatus main body 100 is configured to receive the ECG data obtained by the Holter ECG probes 111 and to generate an ultrasound images by using the reflected-wave signal received by the ultrasound probe 101.

Because the diagnosis apparatus 1 is configured so as to be attachable to the subject P, the diagnosis apparatus 1 is able to obtain the ECG data and the ultrasound images from the subject P, while the subject P is leading a daily life. In particular, because the ultrasound probe 101 according to the first embodiment is configured so as to be thin and have a plate-like shape, it is possible to fix the ultrasound probe 101 onto the subject P.

Next, the shape of the ultrasound probe 101 according to the first embodiment will be explained, with reference to FIGS. 3 to 5. FIG. 3 is a drawing for schematically illustrating an exterior appearance of the ultrasound probe 101 according to the first embodiment being attached to the subject P. FIG. 3 illustrates an exemplary side view of the subject P to whom the ultrasound probe 101 is attached.

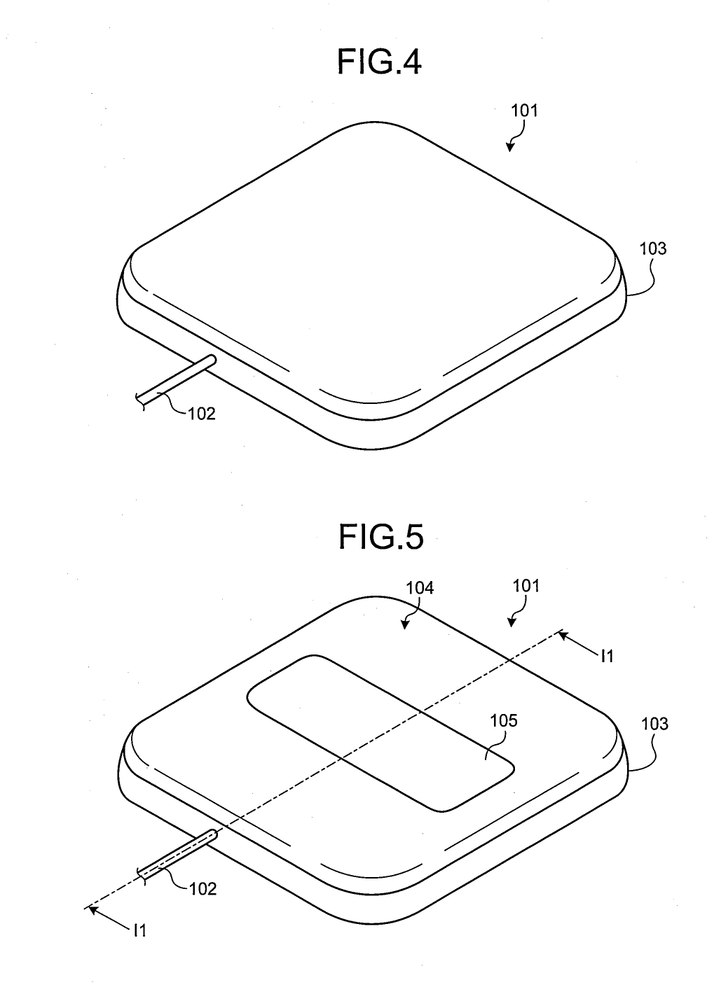

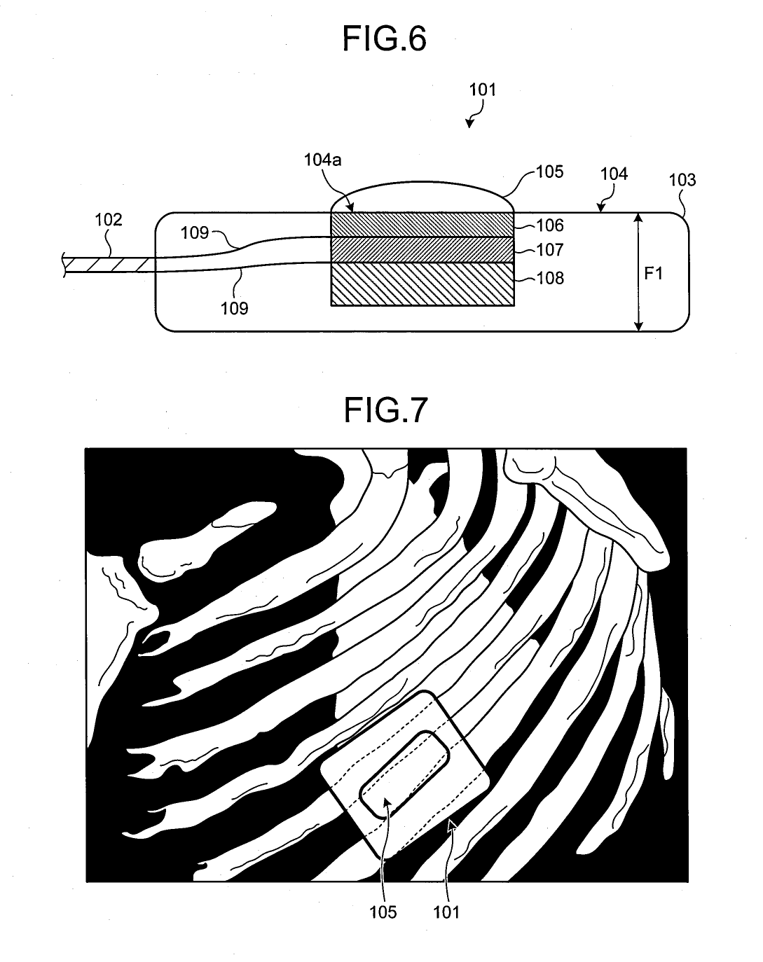

In the example shown in FIG. 3, the ultrasound probe 101 is attached to the body surface of the subject P in the vicinity of the chest and is connected, via the cable 102, to the apparatus main body 100 attached to the vicinity of the waist of the subject P. The ultrasound probe 101 is fixed onto the subject P by a fixing-purpose belt, an adhesive pad, or the like. For example, as shown with the subject P in FIG. 1, the ultrasound probe 101 may be fixed onto the subject P by using a fixing-purpose belt configured with an elastic member that closely adheres to the body surface. In another example, the ultrasound probe 101 may be fixed onto the subject P via an adhesive pad that has adhesiveness and is applied to the subject. The ultrasound probe 101 does not have any protruding grips used by a medical doctor or the like for holding the ultrasound probe 101, so that the shape of the ultrasound probe 101 is unlikely to hinder movements of the subject P, even while the ultrasound probe 101 is fixed onto the subject P. This aspect will be explained, with reference to FIGS. 4 and 5.

FIGS. 4 and 5 are enlarged views of exterior appearances of the ultrasound probe 101 according to the first embodiment. FIG. 4 is an enlarged view of an exterior appearance of the ultrasound probe 101 in the direction of an arrow A1 in FIG. 3. FIG. 5 is an enlarged view of an exterior appearance of the ultrasound probe 101 in the direction of an arrow A2 in FIG. 3.

As shown in FIGS. 4 and 5, the ultrasound probe 101 has an exterior case 103 that has a plate-like shape and is hollow. In the example shown in FIGS. 4 and 5, the exterior case 103 has the shape of a substantially rectangular parallelepiped and is formed by using a synthetic resin, for example. More specifically, in the state illustrated in FIG. 4, the exterior case 103 is formed so that the upper and the lower faces each have a predetermined area, whereas the thickness (i.e., the dimension in the height direction) is small. Further, the exterior case 103 is shaped so that the twelve sides of the rectangular parallelepiped are roundish.

Further, as shown in FIG. 5, the ultrasound probe 101 has an opening formed in a contact face 104, which is a face of the exterior case 103 to be brought into contact with the subject P. An acoustic lens 105 is provided in the opening. The acoustic lens 105 is configured to converge the ultrasound waves generated from piezoelectric elements 107 (explained later). This aspect will be explained with reference to FIG. 6.

FIG. 6 is a cross-sectional view of the ultrasound probe 101 at a line I1-I1 in FIG. 5. As shown in FIG. 6, the exterior case 103 of the ultrasound probe 101 according to the first embodiment has an opening 104a formed in the contact face 104, the opening 104a being a hole that has substantially the same shape as that of the lower face of the acoustic lens 105. Further, the acoustic lens 105 is fixed to the opening 104a. In other words, the acoustic lens 105 is provided so that a curved portion thereof is disposed on the exterior case 103, the curved portion being formed so as to have a convex shape that can be fitted to a space between bones, each of which is a projection part of the subject P.

Further, the ultrasound probe 101 is structured so that, when the contact face 104 of the exterior case 103 is considered as the upper face, an acoustic matching layer 106, the piezoelectric elements 107, and a backing member 108 are laminated in the direction from the acoustic lens 105 toward the lower face of the exterior case 103. As mentioned above, the acoustic lens 105 is configured to converge the ultrasound waves. The acoustic matching layer 106 mitigates mismatches of acoustic impedances between the piezoelectric elements 107 and the subject P.

The piezoelectric elements 107 are connected to the cable 102 by electrodes 109 of a flexible cable or the like and are configured to transmit and receive electric signals to and from the apparatus main body 100 via the electrodes 109. The piezoelectric elements 107 generate ultrasound waves based on transmission signals supplied from the apparatus main body 100 and receive reflected-wave signals from the subject P. More specifically, the piezoelectric elements 107 according to the first embodiment generate the ultrasound waves in a substantially thickness direction F1 of the exterior case 103. Although not shown in the drawing, the piezoelectric elements 107 include two or more piezoelectric elements each of which generates an ultrasound wave and receives a reflected-wave signal. It is assumed that, in the example illustrated in FIG. 6, a plurality of piezoelectric vibrators are arranged in a row. Accordingly, the ultrasound probe 101 described above corresponds to a one-dimensional ultrasound probe. The backing member 108 is configured to prevent the ultrasound waves from propagating rearward (toward the lower face of the exterior case 103) from the piezoelectric elements 107.

For example, when an ultrasound wave is transmitted from the ultrasound probe 101 to the subject P, the transmitted ultrasound wave is repeatedly reflected on a surface of discontinuity of acoustic impedances at a tissue in the body of the subject and is received as a reflected-wave signal by the piezoelectric elements 107 included in the ultrasound probe 101. The amplitude of the received reflected-wave signal is dependent on the difference between the acoustic impedances on the surface of discontinuity on which the ultrasound wave is reflected. When the transmitted ultrasound pulse is reflected on the surface of a flowing bloodstream or a cardiac wall, the reflected-wave signal is, due to the Doppler effect, subject to a frequency shift, depending on a velocity component of the moving members with respect to the ultrasound wave transmission direction. Further, the reflected-wave signal received by the ultrasound probe 101 is transmitted to the apparatus main body 100 via the cable 102. By using the reflected-wave signals received from the ultrasound probe 101, the apparatus main body 100 generates the ultrasound image of the subject P.

As explained above, the ultrasound probe 101 according to the first embodiment includes the plate-like exterior case 103 as illustrated in the examples in FIGS. 3 to 6. Further, the acoustic lens 105 is provided on the contact face 104 of the exterior case 103 that is brought into contact with the subject P. The exterior case 103 has, on the inside thereof, the piezoelectric elements 107 that generate the ultrasound waves emitted in the substantially thickness direction F1 of the exterior case 103, via the acoustic lens 105. Because the ultrasound probe 101 is thin and has a plate-like shape, the ultrasound probe 101 can easily be fixed onto the subject P and is unlikely to hinder movements of the subject P, even while being fixed onto the subject P.

The ultrasound waves emitted from the ultrasound probe 101 described above are substantially totally reflected by the bones and the like inside the subject P. For this reason, even if the user wishes to have an ultrasound image of the heart generated, there is a possibility that the heart may not properly be rendered in the ultrasound image when a bone is positioned between the ultrasound probe 101 and the heart serving as the image taking target. Consequently, when an ultrasound image of the chest of the subject P is to be generated as described in the exemplary embodiment above, it is desirable to arrange the ultrasound waves emitted from the ultrasound probe 101 so as to arrive at the heart or the like while avoiding the ribs of the subject P. For this reason, it is desirable to arrange the acoustic lens 105 included in the ultrasound probe 101 described above so as to have a convex shape that fits an intercostal region of the subject P. This aspect will be explained, with reference to FIG. 7.

FIG. 7 is a schematic drawing of a state of the ultrasound probe 101 fixed in an intercostal region. To clearly indicate the shape of the acoustic lens 105, FIG. 7 illustrates the state as if the acoustic lens 105 was directly fitted to the intercostal region of the subject P. However, in actuality, the ultrasound probe 101 is adhered to the body surface of the subject P and is not in direct contact with the ribs. As shown in the example in FIG. 6, the acoustic lens 105 is formed so as to have a convex shape curving away from the contact face 104 of the exterior case 103. In this situation, as shown in the example in FIG. 7, the acoustic lens 105 is formed so that the curved portion thereof has a shape that can be fitted to an intercostal region of the subject P. With this arrangement, when the ultrasound probe 101 is fixed onto the subject P in such a manner that the acoustic lens 105 is positioned in the intercostal region, the ultrasound waves emitted from the ultrasound probe 101 are able to advance while avoiding the ribs. As a result, the apparatus main body 100 is able to generate an ultrasound image in which the heart or the like surrounded by the ribs are rendered, by using the reflected-wave signals received by the ultrasound probe 101. Further, the acoustic lens 105 having the convex shape that fits the intercostal region can easily be fitted to the intercostal region. Consequently, the configuration makes it easy to fix the ultrasound probe 101 onto the subject P.

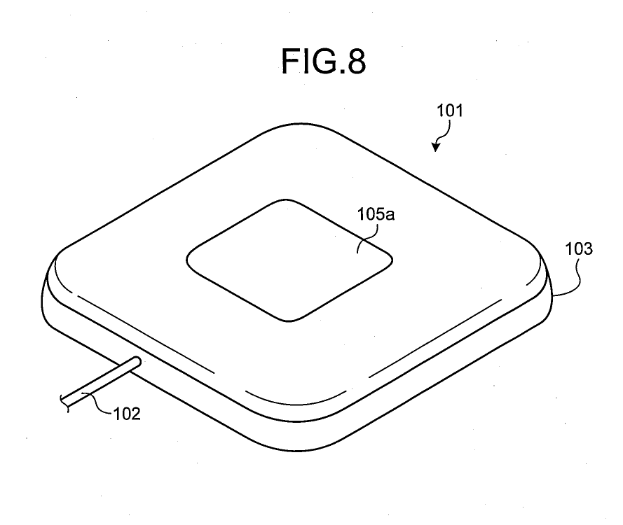

Further, in the first embodiment described above, the example is explained in which the ultrasound probe 101 is a one-dimensional ultrasound probe in which the plurality of piezoelectric vibrators are arranged in a row. However, it is acceptable to configure the ultrasound probe 101 with a two-dimensional ultrasound probe in which a plurality of piezoelectric vibrators are two-dimensionally arranged in a grid formation. FIG. 8 is an enlarged view of an exterior appearance of a two-dimensional ultrasound probe 101 according to the first embodiment. FIG. 8 corresponds to FIG. 5. In the two-dimensional ultrasound probe 101, because the plurality of piezoelectric vibrators are two-dimensionally arranged in the grid formation, an acoustic lens 105a is provided of which the dimensions in the length direction and the width direction are substantially equal, as shown in the example in FIG. 8. It is also desirable to arrange the acoustic lens 105a so as to have a convex shape that fits an intercostal region of the subject P.

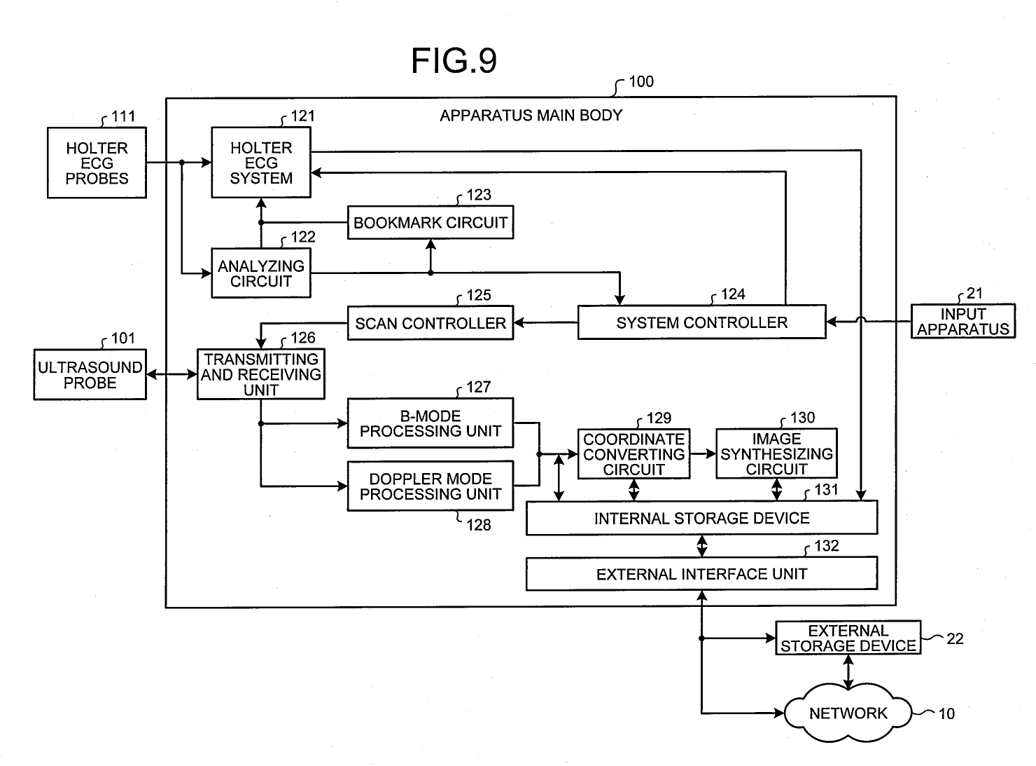

Next, the apparatus main body 100 according to the first embodiment will be explained, with reference to FIG. 9. FIG. 9 is a diagram of an exemplary configuration of the apparatus main body 100 according to the first embodiment. The apparatus main body 100 shown in FIG. 9 has a battery (not shown) or the like installed therein and is configured to operate on the battery. As shown in FIG. 9, the apparatus main body 100 according to the first embodiment has connected thereto the ultrasound probe 101, the Holter ECG probes 111, and an input apparatus 21.

The input apparatus 21 is configured with input devices such as a panel switch, a touch command screen, a trackball, a button, and/or the like. These input devices are provided on a lateral face of the apparatus main body 100, for example. The apparatus main body 100 receives an operation instruction from a user (e.g., the subject P) via the input apparatus 21.

Further, the apparatus main body 100 is connected to the network 10 and an external storage device 22. In the first embodiment, it is assumed that the apparatus main body 100 is wirelessly connected to the network 10 and the external storage device 22. The external storage device 22 is, for example, the server apparatus 12 installed in the hospital as illustrated in FIG. 1 or a storage server connected to the server apparatus 12.

Further, as shown in FIG. 9, the apparatus main body 100 includes a Holter ECG system 121, an analyzing circuit 122, a bookmark circuit 123, a system controller 124, a scan controller 125, a transmitting and receiving unit 126, a B-mode processing unit 127, a Doppler mode processing unit 128, a coordinate converting circuit 129, an image synthesizing circuit 130, an internal storage device 131, and an external interface unit 132.

The Holter ECG probes 111 obtain the ECG data by detecting the small electric signals from the inside of the subject P, while being fixed to the body surface of the subject P by the adhesive pad or the like. The Holter ECG system 121 receives the ECG data obtained by the Holter ECG probes 111. Further, the Holter ECG system 121 stores the ECG data into the internal storage device 131. The Holter ECG system 121 according to the first embodiment is configured to constantly receive the ECG data from the Holter ECG probes 111 and to accumulate the received ECG data into the internal storage device 131.

The analyzing circuit 122 receives the ECG data from the Holter ECG probes 111 and judges whether any abnormality is occurring in the subject P by analyzing the received ECG data in a real-time manner. Further, when having determined that there is a possibility that an abnormality may be occurring in the subject P as a result of the analysis, the analyzing circuit 122 transmits an abnormality occurrence notification to the bookmark circuit 123 and to the system controller 124.

The analyzing process performed by the analyzing circuit 122 can be explained as follows: For example, the analyzing circuit 122 obtains, from the ECG data, a P-wave, QRS waves (a Q-wave, an R-wave, and an S-wave), and a T-wave representing a waveform of cardiac cycles and judges whether an abnormality is occurring in the subject P by using these waves. For example, a time period between the Q-wave and the S-wave denotes a ventricular systolic period, whereas a time period between the S-wave and the T-wave denotes a ventricular diastolic period. Thus, the analyzing circuit 122 judges whether the subject P is suspected to have an ischemic heart disease or a myocardial infarction, by analyzing the motions of the heart in the S-T period (the time period between the S-wave and the T-wave). As another example, a section where the waveform is horizontal at 0 mv can be observed in an S-T period. Angina pectoris makes the horizontal portion lower than that in a normal state, whereas a myocardial infarction makes the horizontal portion higher. Thus, by analyzing the S-T period, the analyzing circuit 122 judges whether the subject P is suspected to have angina pectoris.

When having received an abnormality occurrence notification from the analyzing circuit 122, the bookmark circuit 123 stores therein an abnormality occurrence time indicating the time at which the abnormality occurrence notification was received. For example, the bookmark circuit 123 stores the abnormality occurrence time into a predetermined storage memory as a log. In addition, the bookmark circuit 123 may add the abnormality occurrence time, as a piece of data, to the ECG data from which the abnormality was detected by the analyzing circuit 122.

The system controller 124 is configured by using an electronic circuit such as a Central Processing Unit (CPU) or a Micro Processing Unit (MPU) or an integrated circuit such as an Application Specific Integrated Circuit (ASIC) or a Field Programmable Gate Array (FPGA) and is configured to exercise overall control of the processes performed by the apparatus main body 100. Although the controlling lines are not illustrated in FIG. 9, the system controller 124 controls processes performed by functional units in the apparatus main body 100 by transmitting control signals to the functional units.

When having received an abnormality occurrence notification from the analyzing circuit 122, the system controller 124 according to the first embodiment controls the scan controller 125 so as to perform a scanning process using the ultrasound probe 101 until a predetermined period of time (e.g., one second, two seconds, or five seconds) has elapsed since the time at which the abnormality occurrence notification is received.

By controlling the transmitting and receiving unit 126, the scan controller 125 causes the ultrasound probe 101 to start a scan. In this situation, the scan controller 125 controls the transmitting and receiving unit 126 so as to perform the scan for the time period specified by the system controller 124.

The transmitting and receiving unit 126 performs an ultrasound transmitting and receiving process. More specifically, to transmit ultrasound waves, the transmitting and receiving unit 126 causes a pulser therein to sequentially generate high-voltage pulses in correspondence with predetermined delay periods. When the high-voltage pulses are sequentially applied to the vibrator cells of the piezoelectric elements 107 included in the ultrasound probe 101, an ultrasound wave is generated in each of the vibrator cells.

Further, to receive the ultrasound waves, the vibrator cells of the piezoelectric elements 107 within the ultrasound probe 101 receive the reflected waves of the ultrasound beams, so that reception signals corresponding to a plurality of channels are input to the transmitting and receiving unit 126. After a gain correcting process is applied to the reception signals by a pre-amplifier, the transmitting and receiving unit 126 performs an Analog/Digital (A/D) conversion thereon. Subsequently, after performing delay control and an adding process (a phase-matching addition) on the signals resulting from the A/D conversion for each of the channels in correspondence with each reception focus position, the transmitting and receiving unit 126 generates reflected-wave data by controlling a signal bandwidth by using a quadrature detection and a bandwidth limiting filter and further transmits the generated reflected-wave data to the B-mode processing unit 127 and the Doppler mode processing unit 128.

The B-mode processing unit 127 receives the reflected-wave data from the transmitting and receiving unit 126 and generates data (B-mode data) in which the strength of each signal is expressed by a degree of brightness, by performing a logarithmic amplification, an envelope detection process, and the like on the received reflected-wave data.

The Doppler mode processing unit 128 extracts bloodstreams, tissues, and contrast echo components under the influence of the Doppler effect by performing a frequency analysis so as to obtain velocity information from the reflected-wave data received from the transmitting and receiving unit 126, and further generates data (Doppler data) obtained by extracting bloodstream information such as an average velocity, the dispersion, the power, and the like for a plurality of points.

The B-mode data generated by the B-mode processing unit 127 and the Doppler data generated by the Doppler mode processing unit 128 may be referred to as raw data and are stored in the internal storage device 131. The raw data is also transmitted to the coordinate converting circuit 129.

The coordinate converting circuit 129 converts the raw data received from the B-mode processing unit 127 and the Doppler mode processing unit 128, from a coordinate system used when the data was reception beams, into a rectangular coordinate system used for displaying images.

The image synthesizing circuit 130 stores a B-mode image and a Doppler-mode/color-mode image of which the coordinate systems were changed into the rectangular coordinate system by the coordinate converting circuit 129, into the internal storage device 131 and further performs an image synthesizing process thereon so as to synthesize the images with text information indicating an image acquisition condition or the like. After that, the image synthesizing circuit 130 assigns Red-Green-Blue (RGB) map values thereto. Thus, the image synthesizing circuit 130 generates synthesized images as the ultrasound images.

The internal storage device 131 is a storage device configured with a Random Access Memory (RAM), a flash memory, a flash Solid State Drive (SSD), or the like. The internal storage device 131 stores therein the raw data generated by the B-mode processing unit 127 and the Doppler mode processing unit 128, as well as the ultrasound images and the like generated by the image synthesizing circuit 130.

The external interface unit 132 transmits and receives various types of data to and from external apparatuses via wireless communications. More specifically, the system controller 124 has a wireless communication function and is capable of storing the raw data, the ultrasound images, and the like stored in the internal storage device 131 into the external storage device 22.

In this situation, when the system controller 124 according to the first embodiment has received the abnormality occurrence notification from the analyzing circuit 122 and has controlled the scan controller 125 so as to perform the scanning process for the predetermined period of time, the system controller 124 stores the abnormality occurrence time recorded by the bookmark circuit 123, the ECG data from which the abnormality was detected by the analyzing circuit 122, and the ultrasound image generated as a result of controlling the scan controller 125, into the internal storage device 131 while keeping these items in correspondence with one another. Further, the system controller 124 transmits the group of data that is stored in the internal storage device 131 and in which the abnormality occurrence time, the ECG data, and the ultrasound image are kept in correspondence with one another to the server apparatus 12. The system controller 124 may regularly obtain such a group of data from the internal storage device 131 and transmit the obtained group of data to the server apparatus 12 or may transmit such a group of data to the server apparatus 12 every time an abnormality is detected by the analyzing circuit 122.

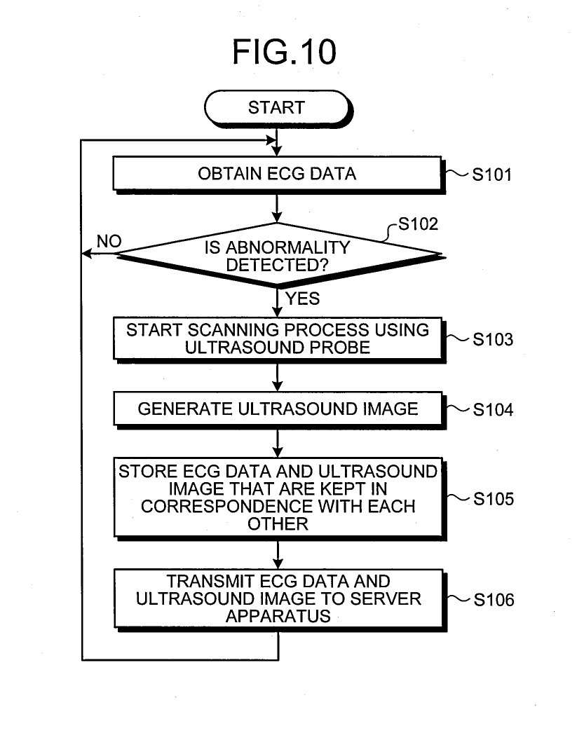

Next, a processing procedure performed by the diagnosis apparatus 1 according to the first embodiment will be explained, with reference to FIG. 10. FIG. 10 is a flowchart of the processing procedure performed by the diagnosis apparatus 1 according to the first embodiment.

As shown in FIG. 10, the apparatus main body 100 of the diagnosis apparatus 1 sequentially obtains pieces of ECG data of the subject P via the Holter ECG probes 111 (step S101). After that, the analyzing circuit 122 included the apparatus main body 100 judges whether an abnormality is occurring in the subject P, by analyzing the pieces of ECG data sequentially obtained (step S102). Subsequently, as long as no abnormality is detected in the subject P by the analyzing circuit 122 (step S102: No), the apparatus main body 100 sequentially obtains pieces of ECG data of the subject P via the Holter ECG probes 111 (step S101).

On the contrary, when an abnormality is detected in the subject P by the analyzing circuit 122 (step S102: Yes), the system controller 124 included in the apparatus main body 100 starts a scanning process using the ultrasound probe 101 by controlling the scan controller 125 (step S103). As a result, the ultrasound probe 101, the transmitting and receiving unit 126, the B-mode processing unit 127, the Doppler mode processing unit 128, the coordinate converting circuit 129, the image synthesizing circuit 130, and the like perform processes, and the apparatus main body 100 thus generates an ultrasound image (step S104).

After that, the system controller 124 stores the ECG data from which the abnormality was detected at step S102 and the ultrasound image generated at step S104, into the internal storage device 131, while keeping these items in correspondence with each other (step S105). Subsequently, the system controller 124 transmits the set that is made up of the ECG data and the ultrasound image and is stored in the internal storage device 131 to the server apparatus 12 (step S106).

As explained above, according to the first embodiment, it is possible to attach the ultrasound probe 101 to the subject P.

Further, according to the first embodiment, it is possible to examine the subject in an integral manner while using the ECGs and the ultrasound images. Thus, even if the waveforms of the ECGs are disturbed by the posture changes and/or the body movements of the subject P, it is possible to improve the level of precision of the diagnosis made by the medical doctor.

In the first embodiment, the example is explained in which the diagnosis apparatus 1 generates an ultrasound image when an abnormality is detected by analyzing the ECG. However, it is acceptable to configure the diagnosis apparatus 1 so as to generate an ultrasound image even if no abnormality is detected in an analysis result of the ECG. For example, it is acceptable to configure the diagnosis apparatus 1 so as to start the scanning process using the ultrasound probe 101 and to generate an ultrasound image every time a predetermined period of time has elapsed.

In another example, it is also acceptable to configure the diagnosis apparatus 1 so as to generate an ultrasound image at a specific time. For example, generally speaking, it is known that arrhythmias and coronary spastic angina involving a spasm of a coronary artery often occur at night or in the early morning regardless of physical exertion. Thus, it is sometimes difficult to make a diagnosis from an ECG test or a stress ECG test performed at a hospital. Consequently, it is acceptable to configure the diagnosis apparatus 1 so that the process of causing the ultrasound probe 101 to start the scanning process is performed in a concentrated manner at night and in the early morning. With this arrangement, the diagnosis apparatus 1 may be able to generate an ultrasound image that makes it possible to diagnose coronary spastic angina or the like of the subject P.

Further, when the diagnosis apparatus 1 is used for the purpose of regularly generating ultrasound images or for the purpose of generating ultrasound images in specific periods of time as described in the examples above, the diagnosis apparatus 1 does not necessarily have to include the ECG system. More specifically, the diagnosis apparatus 1 does not necessarily have to include the Holter ECG probes 111, the Holter ECG system 121, the analyzing circuit 122, and the bookmark circuit 123 shown in FIG. 9.

Further, in the first embodiment described above, the example is explained in which, if occurrence of an abnormality is detected by analyzing an ECG, the diagnosis apparatus 1 performs the scanning process using the ultrasound probe 101, until the predetermined period of time has elapsed since the abnormality occurrence time. However, it is also acceptable to configure the diagnosis apparatus 1 so as to, if occurrence of an abnormality is detected, perform the scanning process using the ultrasound probe 101 until a predetermined number of ultrasound images have been generated.

Further, in the first embodiment described above, it is acceptable to configure the diagnosis apparatus 1 so as to identify cardiac phases by analyzing the ECG and to intermittently perform the scanning process using the ultrasound probe 101 at times in a specific cardiac phase. Further, it is acceptable to configure the diagnosis apparatus 1 so as to transmit the intermittently-generated ultrasound images and such ECGs that were obtained when the ultrasound images were generated, to the server apparatus 12. In that situation, it is acceptable to configure the server apparatus 12 so as to analyze, in a real-time manner, the ultrasound images and the ECGs transmitted from the diagnosis apparatus 1 and so as to, if an abnormality is detected in motions of the cardiac walls or the like, store an abnormality occurrence time into a predetermined storage memory as a log.

Further, in the first embodiment described above, it is acceptable to configure the diagnosis apparatus 1 so as to obtain volume data, which is three-dimensional medical image data, if the ultrasound probe 101 is configured with a two-dimensional ultrasound probe as illustrated in FIG. 8.

Further, in the first embodiment described above, it is acceptable to configure the system controller 124 included in the diagnosis apparatus 1 so as to send, via e-mail for example, an alert to a portable terminal or the like that is held by a medical doctor or the like, when the number of times an abnormality is detected by the analyzing circuit 122 has exceeded a predetermined value or when the analyzing circuit 122 has kept detecting abnormalities for a predetermined period of time.

Further, in the first embodiment described above, it is acceptable to configure the diagnosis apparatus 1 so as to include a wrist-watch-style pulse measuring apparatus configured to obtain a pulse rate of the subject P, instead of the Holter ECG probes 111. In that situation, the analyzing circuit 122 determines that an abnormality has occurred in the subject P when, for example, the pulse rate is not in a predetermined threshold range.

Further, in the first embodiment described above, it is acceptable to configure the transmitting and receiving unit 126, the B-mode processing unit 127, the Doppler mode processing unit 128, the coordinate converting circuit 129, the image synthesizing circuit 130, and the like illustrated in FIG. 9 so as to operate in a power-saving mode (i.e., a standby state) and so as to, when an abnormality is detected by the analyzing circuit 122, exit the power-saving mode (i.e., the standby state) and operate in a normal power-supply mode, according to the control of the system controller 124.

Further, in the first embodiment described above, it is acceptable to configure the diagnosis apparatus 1 so as not to generate the ultrasound images, but so as to transmit the reflected-wave signals received by the ultrasound probe 101 to the server apparatus 12. In that situation, the ultrasound probe 101 does not necessarily have to include the B-mode processing unit 127, the Doppler mode processing unit 128, the coordinate converting circuit 129, and the image synthesizing circuit 130 illustrated in FIG. 9. With this arrangement, it is possible to make the ultrasound probe 101 more compact. Further, in that situation, the server apparatus 12 has functions equivalent to those of the B-mode processing unit 127, the Doppler mode processing unit 128, the coordinate converting circuit 129, and the image synthesizing circuit 130 illustrated in FIG. 9 and generates ultrasound images by using the reflected-wave signals received from the diagnosis apparatus 1.

In the examples described above, it is acceptable to configure the diagnosis apparatus 1 so as to perform up to the process of generating the raw data from the reflected-wave signals received by the ultrasound probe 101 and to transmit the generated raw data to the server apparatus 12. In that situation, the ultrasound probe 101 does not necessarily have to include the coordinate converting circuit 129 and the image synthesizing circuit 130 illustrated in FIG. 9. Further, in that situation, the server apparatus 12 has functions equivalent to those of the coordinate converting circuit 129 and the image synthesizing circuit 130 illustrated in FIG. 9 and generates ultrasound images by using the raw data received from the diagnosis apparatus 1. In this example, because the raw data generated by the B-mode processing unit 127 or the Doppler mode processing unit 128 is smaller in data size than the reflected-wave signals, it is possible to prevent the communication bandwidth between the diagnosis apparatus 1 and the access point 11 from being congested. Similarly, it is also possible to prevent the communication bandwidth between the access point 11 and the server apparatus 12 from being congested.

Further, in the examples described above, it is acceptable to configure the diagnosis apparatus 1 so that the transmission is directed to a desktop personal computer, a notebook personal computer, a tablet personal computer, a portable terminal, or the like used by a medical doctor, a nurse, or the like. Further, it is also acceptable to configure the diagnosis apparatus 1 so as to transmit only the ECGs or only the ultrasound images to the server apparatus 12 or to a personal computer or the like used by a medical doctor or the like, instead of transmitting the sets each made up of an ECG and an ultrasound image. Further, it is also acceptable to configure the diagnosis apparatus 1 so as to transmit sets made up of ECGs and ultrasound images obtained before and after the time at which an abnormality is detected by the analyzing circuit 122.

In the first embodiment, the shape of the ultrasound probe 101 that is thin and has a plate-like shape was explained, with reference to FIGS. 3 to 8. However, the shape of an ultrasound probe connected to the diagnosis apparatus 1 is not limited to the one described in the first embodiment. In a second embodiment, other exemplary shapes of the ultrasound probe will be explained.

Sloped Face

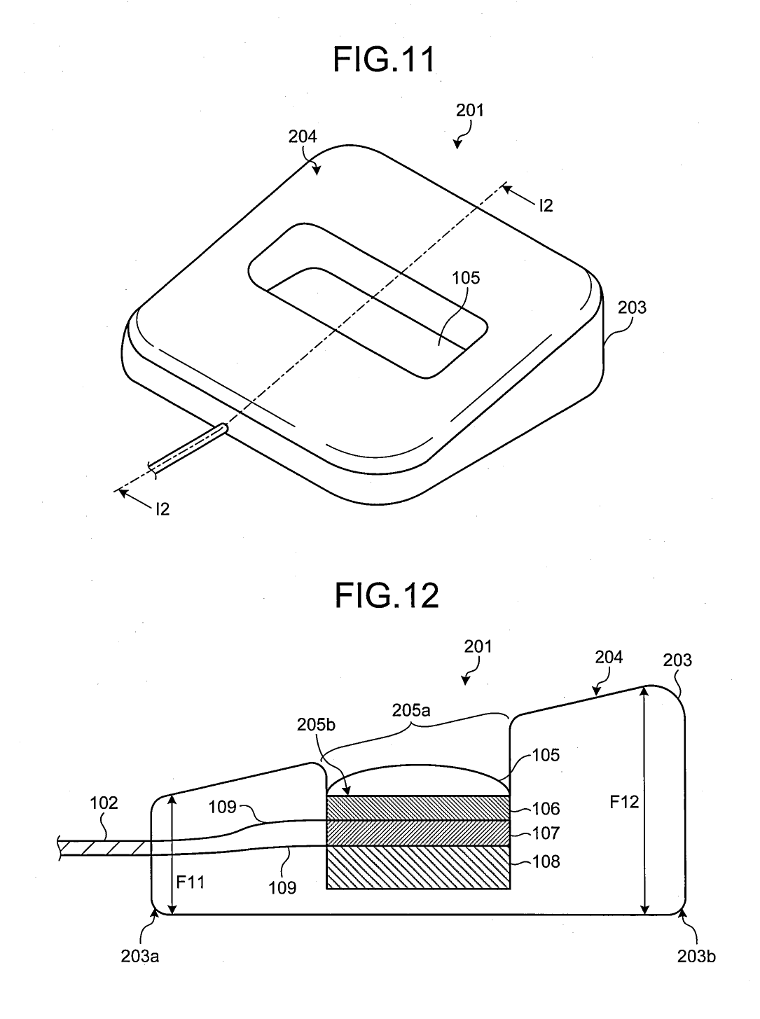

In the first embodiment, the example is explained in which the ultrasound probe has the exterior case 103 having the shape of a substantially rectangular parallelepiped in which the upper face and the lower face are positioned substantially parallel to each other. However, it is acceptable to configure an ultrasound probe so as to have an exterior case in which the two faces are not positioned parallel to each other. This aspect will be explained with reference to FIGS. 11 and 12. FIG. 11 is an enlarged view of an exterior appearance of an ultrasound probe 201 according to a first modification example. FIG. 12 is a cross-sectional view of the ultrasound probe 201 at a line I2-I2 in FIG. 11.

As shown in FIG. 11, the ultrasound probe 201 according to the first modification example is shaped so that a contact face 204 to be brought into contact with the subject P is a sloped face. More specifically, in the example shown in FIG. 12, an exterior case 203 of the ultrasound probe 201 is shaped so that a thickness F12 of a lateral face 203b is larger than a thickness F11 of a lateral face 203a to which the cable 102 is connected, the lateral face 203b being positioned opposite to the lateral face 203a. The thickness of the exterior case 203 increases from the lateral face 203a toward the lateral face 203b. In other words, in the example shown in FIG. 11, the ultrasound probe 201 according to the first modification example is configured so that an opening 205a having a bottom 205b that is substantially parallel to the lower face of the ultrasound probe 201 is formed in a part of the contact face 204, which is formed as the sloped face that is not positioned parallel to the lower face. The acoustic lens 105 is fixed to the bottom 205b of the opening 205a.

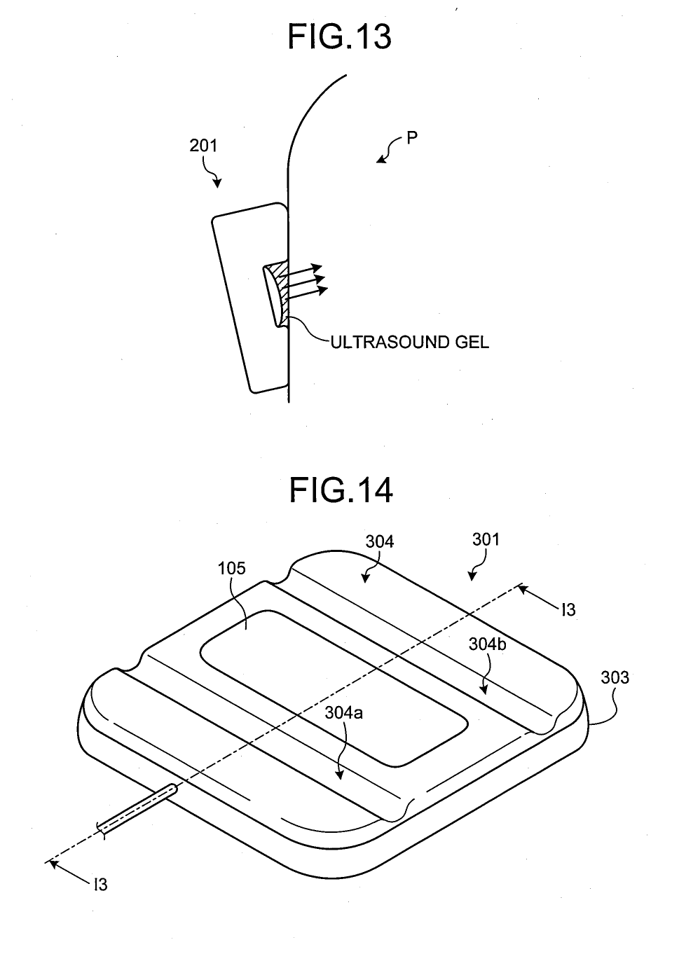

FIG. 13 is a drawing of an exterior appearance of the ultrasound probe 201 according to the first modification example being fixed onto the subject P. As shown in FIG. 13, when the ultrasound probe 201 according to the first modification example is fixed onto the subject P, the direction of the ultrasound waves emitted through the acoustic lens 105 is tilted in accordance with the slope of the contact face 204. As a result, even if the piezoelectric elements included therein are not of a swingable type, the ultrasound probe 201 according to the first modification example is able to transmit the ultrasound waves in the directions other than the direction substantially perpendicular to the body surface, in accordance with the angle formed by the upper and the lower faces of the exterior case 203. As shown in FIG. 13, ultrasound gel is applied to the space between the subject P and the acoustic lens 105 of the ultrasound probe 201 so as to fill in the space.

Grooves

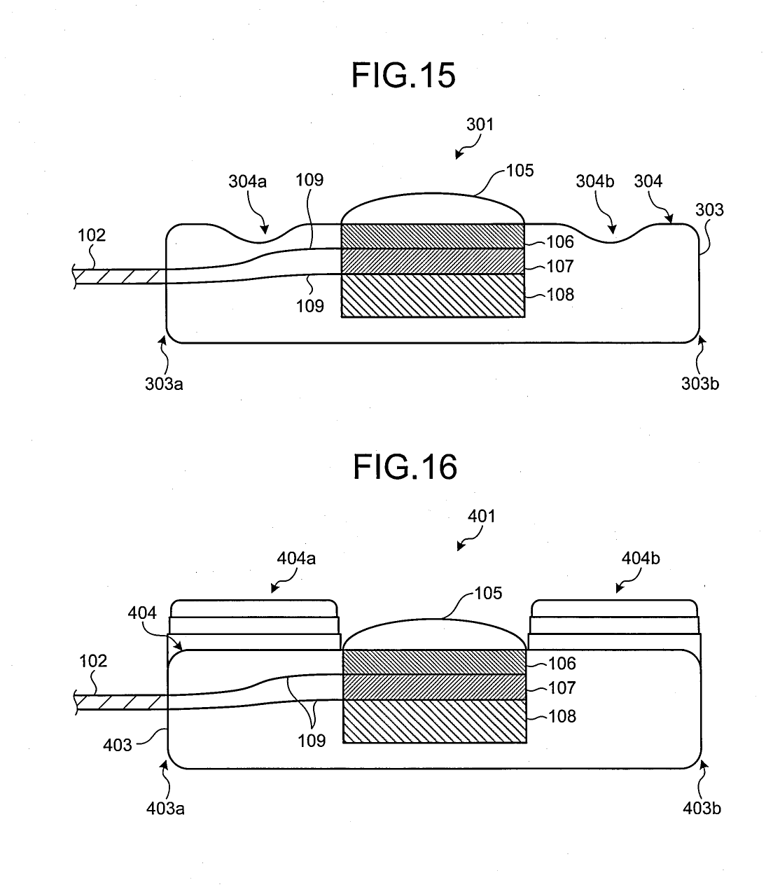

In the first embodiment described above, the example is explained in which the ultrasound probe has the plate-like exterior case 103 having the shape of a substantially rectangular parallelepiped in which the upper face and the lower face are positioned substantially parallel to each other. However, it is also acceptable to configure an ultrasound probe so as to have an exterior case in which concave portions to be engaged with projection parts (e.g., bones) of the subject P are formed in the contact face which is one of the upper and the lower faces that has the acoustic lens 105 provided thereon. This aspect will be explained with reference to FIGS. 14 and 15. FIG. 14 is an enlarged view of an exterior appearance of an ultrasound probe 301 according to a second modification example. FIG. 15 is a cross-sectional view of the ultrasound probe 301 at a line I3-I3 in FIG. 14.

As shown in FIGS. 14 and 15, the ultrasound probe 301 according to the second modification example has an exterior case 303 in which concave portions 304a and 304b, which are substantially linear grooves, are formed in a contact face 304 to be brought into contact with the subject P. In the example shown in FIGS. 14 and 15, the exterior case 303 has the concave portions 304a and 304b formed so that the acoustic lens 105 is interposed therebetween. More specifically, in the example shown in FIG. 15, the exterior case 303 of the ultrasound probe 301 has the concave portion 304a formed so as to be positioned between a lateral face 303a to which the cable 102 is connected and the acoustic lens 105 and has the concave portion 304b formed so as to be positioned between a lateral face 303b positioned opposite to the lateral face 303a and the acoustic lens 105. The concave portions 304a and 304b are grooves that cave into the contact face 304 in the direction toward the lower face (i.e., the bottom) and are configured to be engaged with the projection parts of the subject P.

Because the concave portions 304a and 304b are shaped so as to be easily fitted to an intercostal region, it is possible to easily fix the ultrasound probe 301 onto the subject P. More specifically, because the ultrasound probe 301 has the exterior case 303 in which the concave portions 304a and 304b are formed on either side of the acoustic lens 105, the concave portions 304a and 304b are positioned at the ribs of the subject P, in the example illustrated in FIG. 7. Consequently, it is possible to easily fix the ultrasound probe 301 onto the subject P. As a result, the configuration makes it possible to regularly take images of a fixedly-selected site (e.g., the heart) inside the subject P.

Adaptors

In the first embodiment described above, the example is explained in which the ultrasound probe has the plate-like exterior case 103 having the shape of a substantially rectangular parallelepiped in which the upper face and the lower face are positioned substantially parallel to each other. However, it is also acceptable to configure an ultrasound probe so as to have an exterior case provided with expandable members that are expandable in a direction away from the contact face (i.e., a plurality of plate-like adhesive members among which one or more are piled up). This aspect will be explained with reference to FIG. 16. FIG. 16 is a cross-sectional view of an ultrasound probe 401 according to a third modification example.

As shown in FIG. 16, the ultrasound probe 401 according to the third modification example is configured so that adaptors 404a and 404b are provided as the expandable members that are expandable in the direction away from a contact face 404, on at least such an area of the contact face 404 of an exterior case 403 that excludes a central part. In the example shown in FIG. 16, the adaptors 404a and 404b are provided on the contact face 404 of the exterior case 403 so that the acoustic lens 105 is interposed therebetween. More specifically, in the example illustrated in FIG. 16, the adaptor 404a is provided on the contact face 404 of the exterior case 403 so as to be positioned between a lateral face 403a of the exterior case 403 to which the cable 102 is connected and the acoustic lens 105. The adaptor 404b is provided on the contact face 404 of the exterior case 403 so as to be positioned between a lateral face 403b positioned opposite to the lateral face 403a and the acoustic lens 105.

The adaptors 404a and 404b are members that are capable of freely expanding and contracting in the thickness direction of the exterior case 403. For example, as shown in the example in FIG. 16, the adaptors 404a and 404b are each structured by joining together a plurality of circular columnar members having mutually-different diameters in an expandable/contractible manner. In the example in FIG. 16, the adaptors 404a and 404b are in an expanded state in which the circular columnar members are piled on top of one another with the smallest overlapping area.

Even if the piezoelectric elements included therein are not of a swingable type, the ultrasound probe 401 according to the third modification example is able to transmit the ultrasound waves in the directions other than the direction substantially perpendicular to the body surface, in accordance with the expansion/contraction state of the adaptors 404a and 404b. Further, by changing the expansion/contraction state of the adaptors 404a and 404b, it is possible to adjust the emission directions of the ultrasound waves of the ultrasound probe 401. Like in the example shown in FIG. 13, ultrasound gel is applied to the space between the subject P and the acoustic lens 105 of the ultrasound probe 401 so as to fill in the space.

Further, although not shown in the drawings, it is acceptable to configure the ultrasound probe 401 according to the third modification example so as to be provided with another member that is expandable/contractible or an elastic member, instead of the adaptors 404a and 404b. With this arrangement, when the ultrasound probe 401 is adhered, with pressure, to the body surface of the subject P by a fixation band or the like, it is possible to adjust the angle formed by the contact face 404 and the body surface by changing the shape of the expandable/contractible member or the like. Consequently, like in the example in FIG. 13, the ultrasound probe 401 is able to transmit the ultrasound waves in the directions other than the direction substantially perpendicular to the body surface. In that situation, to adjust the angle, it is possible to correct the tilt angle by using together elastic members having mutually-different degrees of hardness (i.e., members having mutually-different shape-change ratios). Thus, like in the example in FIG. 16, it is possible to make a fine adjustment so as to be able to transmit the ultrasound waves in desired directions other than the direction substantially perpendicular. Further, like in the example in FIG. 13, ultrasound gel is applied to the space between the subject P and the acoustic lens 105 of the ultrasound probe 401 so as to fill in the space.

Further, although not shown in the drawings, it is also acceptable to configure the ultrasound probe 101 according to the first embodiment in such a manner that adhesive pads having mutually-different thicknesses are pasted on the contact face 104. In this situation also, the ultrasound probe 101 is able to transmit the ultrasound waves in the directions other than the direction substantially perpendicular to the body surface, like in the example in FIG. 13. In addition, it is possible to easily correct the tilt angle by changing the thicknesses of the adhesive pads. Consequently, like in the example in FIG. 16, it is possible to make a fine adjustment so as to be able to transmit the ultrasound waves in desired directions other than the direction substantially perpendicular. Further, like in the example in FIG. 13, ultrasound gel is applied to the space between the subject P and the acoustic lens 105 of the ultrasound probe 101 so as to fill in the space.

The shapes of the ultrasound probes 101, 201, 301, and 401 are not limited to the examples described above. For instance, in the examples described above, the faces (e.g., the contact face) of the exterior case are substantially rectangular. However, the faces of the exterior case may have an arbitrary shape that is circular, oval, trapezoidal, or the like. Further, in the examples described above, the acoustic lens 105 is provided near the center of the contact face. However, it is acceptable to provide the acoustic lens 105 in an area other than the area near the center of the contact face.

Further, in the example shown in FIG. 11, the contact face 204 and the bottom of the exterior case 203 form the predetermined angle therebetween. However, the contact face 204 and the bottom of the ultrasound probe 201 may be positioned substantially parallel to each other. In that situation, the bottom 205b of the opening 205a and the contact face 204 of the exterior case 203 shown in FIG. 12 have such a relationship so as to form a predetermined angle therebetween. Even in that situation, it is possible to transmit the ultrasound waves in the directions other than the direction perpendicular to the body surface.

Further, in the example in FIG. 14, the substantially linear concave portions 304a and 304b are formed in the contact face 304 of the exterior case 303. However, the concave portions 304a and 304b do not necessarily have to be substantially linear and may have any shape as long as the concave portions 304a and 304b are able to engage with projection parts (e.g., bones) of the subject P.

Further, it is effective to apply the ultrasound probes of which the ultrasound transmission directions are controllable as shown in FIGS. 11 to 13 and 16, to a one-dimensional ultrasound probe. In other words, although the ultrasound transmission directions of one-dimensional ultrasound probes are fixed, it becomes possible to control the ultrasound transmission directions by using the structures shown in FIGS. 11 to 13 and 16.

Stationary-Type Apparatus

In the exemplary embodiments explained with reference to FIGS. 1 to 16, it is assumed that the diagnosis apparatus 1 is carried by the subject P. However, it is also acceptable to configure the diagnosis apparatus 1 so as to be of a stationary type that is not carried by the subject P. More specifically, of the diagnosis apparatus 1 described above, the ultrasound probe 101 and the Holter ECG probes 111 may be carried by the subject P, whereas the apparatus main body 100 may be installed in a clinic room or the like without being carried by the subject P.

An exemplary embodiment of the diagnosis apparatus 1 of a stationary type will be explained, while using a stress echo test as an example. In recent years, tests called “stress echo tests” are performed for the purpose of checking for heart diseases such as an ischemic heart disease. A stress echo test is an ultrasound examination performed while stress is applied to the heart, for the purpose of checking for the changes in the motions of myocardia and in the blood flows that cannot be observed while the subject is at rest. Examples of stress echo tests include exercise-stress cardiac echo tests and drug-stress cardiac echo tests. The heart rate and the blood pressure are raised, by prompting the subject to do physical exercise of mutually-different levels of stress in the former example and by changing the amount of the drug (e.g., dobutamine) in stages in the latter example. When the subject is not capable of doing physical exercise, a drug-stress test is performed. However, exercise-stress tests are preferred because exercise-stress tests use no drug and are safer. During an exercise-stress cardiac echo test, after the subject P does physical exercise as described above, the ultrasound probe is pressed against the subject P so as to record ultrasound images in the form of a moving picture or a group of still images for the duration of one heartbeat or longer, and an ECG is also recorded by attaching the ECG probes to the subject P. In this situation, during the exercise-stress cardiac echo test, it is required to record the ultrasound images and the ECG before a predetermined period of time (e.g., 90 seconds) elapses since the end of the physical exercise of the subject P. In other words, the operator such as the medical doctor is required to press the ultrasound probe against the subject P and to attach the ECG probes to the subject P, immediately after the subject P has finished the physical exercise. To record the ultrasound images, it is necessary to press the ultrasound probe against the subject P, while ensuring that the observation target site (e.g., the heart) is irradiated by the ultrasound waves. Consequently, operators who give stress echo tests are required to be highly skillful.

In contrast, when the diagnosis apparatus 1 of a stationary type according to the exemplary embodiment described above is used, the operator is not required to be highly skillful and is able to easily record the ultrasound images and the ECG. More specifically, while the ultrasound probe 101 and the Holter ECG probes 111 according to the embodiment are attached to the subject P, the subject P is prompted to do physical exercise. After that, when the subject P has finished the physical exercise, the operator is able to immediately record the ultrasound images and the ECG of the subject P who has finished the physical exercise, by operating the apparatus main body 100. As explained above, because the ultrasound probe 101 according to the embodiment is fixed onto the subject P while being fitted to the intercostal region or the like, it is possible to prevent the ultrasound probe 101 from making positional shifts even while the subject P is doing the physical exercise. Consequently, the operator is able to attach the ultrasound probe 101 to the subject P before the start of the physical exercise, spending sufficient time to ensure that the observation target site (e.g., the heart) is irradiated by the ultrasound waves. Further, even after the subject P has finished the physical exercise, the operator is able to record the ultrasound images of the observation target without the need to adjust the attachment position of the ultrasound probe 101.

As explained above, even if the apparatus main body 100 is of a stationary type, the diagnosis apparatus 1 described above is able to realize a medical examination such as a stress echo test that has a high level of precision and is efficient, because the ultrasound probe 101 and the Holter ECG probes 111 are fixed onto the subject P. Further, by attaching the ultrasound probe 101 to the same location of the subject P again and again, it is possible to record ultrasound images of the same observation target many times. Thus, it is possible to utilize the diagnosis apparatus 1 described above as an ultrasound diagnosis apparatus having high reproducibility.

It is also acceptable to configure the diagnosis apparatus 1 described above so as to include a plurality of ultrasound probes 101. In that situation, when there are a plurality of observation targets, the operator is able to record a plurality of ultrasound images at once, by attaching the ultrasound probes 101 to the subject P in such a manner that the observation targets are irradiated by the ultrasound waves. For example, during an exercise-stress cardiac echo test, to record ultrasound images on a specific cross-sectional plane of the heart that can be viewed from a plurality of observation positions in intercostal regions such as an apical window and a parasternal window, which are called cardiac acoustic windows, a certain skill is required to be able to press the ultrasound probe against the subject at an appropriate angle in the observation positions, within a predetermined period of time since the end of the physical exercise. In some situations, it may be required to have the subject P do the physical exercise many times. However, because the diagnosis apparatus 1 according to the embodiment is able to record a plurality of ultrasound images at once, it is possible to realize a stress echo test without the need to have the subject P do the physical exercise many times. If the reflected waves of the ultrasound waves from the probes interfere with one another, time-difference control is exercised so that the transmissions and the receptions are performed while sequentially switching among the probes.

Automatic Exercise-Stress Cardiac Echo Test

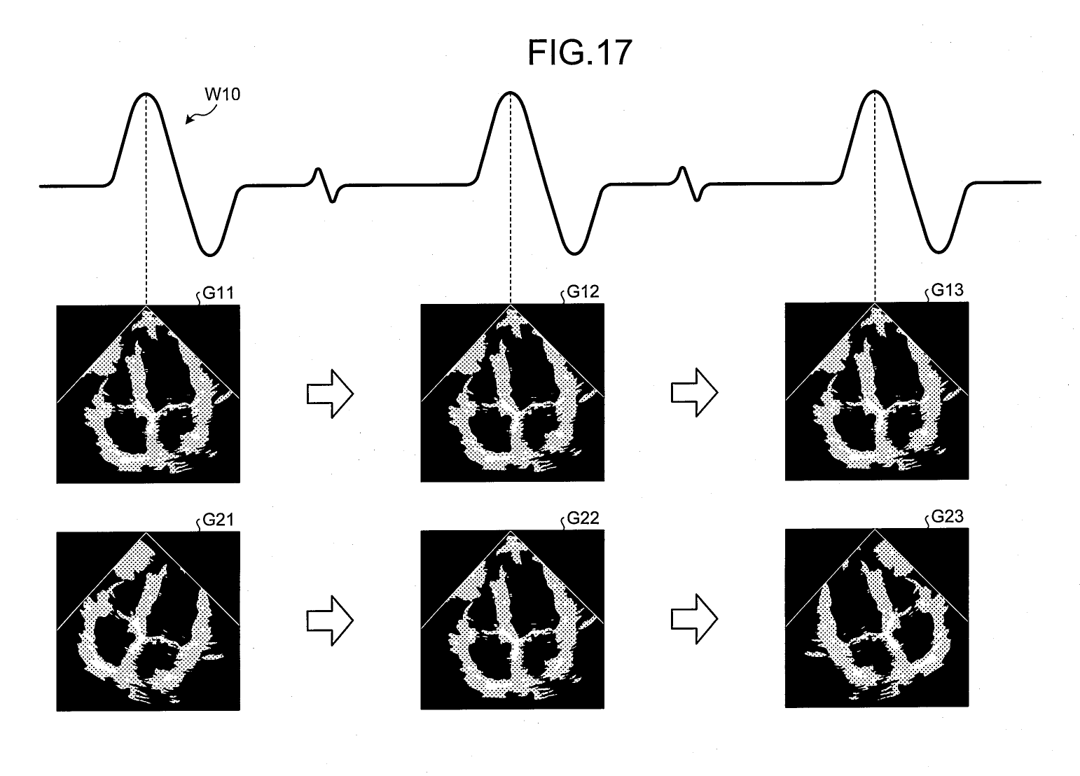

In the description above, the example is explained in which the exercise-stress cardiac echo test is performed by the operator who operates the apparatus main body 100 after the subject P does the physical exercise. However, it is also acceptable to configure the apparatus main body 100 so as to automatically record the ultrasound images and the ECG immediately after the physical exercise by detecting the point in time at which the subject P has finished the physical exercise. This aspect will be specifically explained with reference to FIG. 17. FIG. 17 is a drawing of an example of an exercise-stress cardiac echo test performed by the diagnosis apparatus 1 according to the exemplary embodiment.

In the example shown in FIG. 17, let us assume that the Holter ECG probes 111 have recorded an ECG waveform W10. Also, let us assume that ultrasound images G11 to G13 and G21 and G23 are generated at the times at each of which an R-wave is detected in the ECG waveform W10. The system controller 124 included in the apparatus main body 100 intermittently generates the ultrasound images by controlling the scan controller 125 only at the times when the R-waves are detected in the ECG waveform W10. In the present example, the ultrasound images are sequentially generated at the times at each of which an R-wave is detected. It is, however, acceptable to configure the apparatus main body 100 so as to intermittently generate ultrasound images at the times when the R-waves are detected, after a predetermined period of time (e.g., one minute) has elapsed.

Further, by analyzing the ultrasound images G11 to G13 and G21 to G23, the apparatus main body 100 judges whether the subject P is currently exercising. More specifically, even if the subject P is not exercising, the shape and the position of the heart change due to the expansion and contraction motions of the heart. However, at the times when the R-waves are detected, the shape and the position of the heart are considered to be substantially the same, as long as the subject is not exercising. For this reason, by analyzing (e.g., by applying a cross-correlation process to) a motion vector or the like among the ultrasound images generated at the times when the R-waves are detected, the apparatus main body 100 detects whether the shape and the position of the heart are changing. Further, the apparatus main body 100 determines that the subject P is not exercising if the magnitude of the motion vector is smaller than a predetermined value and determines that the subject P is exercising if the magnitude of the motion vector is equal to or larger than the predetermined value.

For instance, in the example in FIG. 17, let us assume that the ultrasound images G11 to G13 corresponding to the R-waves are consecutively generated. In this example, the positions of the heart rendered in the ultrasound images G11 to G13 are substantially the same as one another. Consequently, because the position of the heart is unchanged at the points in time when the ultrasound images G11 to G13 are generated, the apparatus main body 100 is able to determine that the subject P is not exercising.

As another example, in the example in FIG. 17, let us assume that the ultrasound images G21 to G23 corresponding to the R-waves are consecutively generated. In this example, the positions of the heart rendered in the ultrasound images G21 to G23 are different from one another. Consequently, because the positions of the heart are different among the points in time when the ultrasound images G21 to G23 are generated, the apparatus main body 100 is able to determine that the subject P is exercising.

By analyzing the ultrasound images corresponding to the R-waves in the ECG waveform W10 in this manner, the apparatus main body 100 determines whether the subject P is currently exercising. Further, the apparatus main body 100 generates the ultrasound images consecutively when the subject P has transitioned from an exercising state into a non-exercising state. In other words, the apparatus main body 100 transitions from the state in which the apparatus main body 100 intermittently generates the ultrasound images at the times when the R-waves are detected to the state in which the apparatus main body 100 consecutively generates the ultrasound images regardless of the timing with which the R-waves are detected. In that situation, a moving picture or a group of still images for the duration of one heartbeat or longer is acquired and stored. The user is able to specify, in advance, the acquisition period or the number of heartbeats. Further, it is also judged in parallel, as necessary, whether the subject P is exercising during the acquisition period. When it has been detected that the subject P is exercising during the acquisition period, the moving picture or the group of still images is discarded or information indicating that there have been movements is added thereto, and at the same time, the user is informed by a display on a screen.

With these arrangements, the apparatus main body 100 consecutively generates the ultrasound images automatically, when the subject P who is doing the physical exercise comes to a halt. Thus, the stress echo test is automatically performed without the operator's having to operate the apparatus main body 100.

The process of judging whether the subject P is exercising or not described above may be performed by the system controller 124 included in the apparatus main body 100 or may be performed by a dedicated computer chip or a dedicated computer program included in the apparatus main body 100. Further, in the description above, the example is explained in which the ultrasound images are generated at the times when the R-waves are detected; however, it is acceptable to configure the apparatus main body 100 so as to generate the ultrasound images at the times when other waves such as P-waves, Q-waves, S-waves, T-waves, or U-waves are detected or at the times defined by arbitrary delay periods since an easily-detected wave.

As explained above, according to the first and the second embodiments, it is possible to attach the ultrasound probe to the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
an ultrasound probe of which a contact face to be in contact with a subject for a purpose of adhering thereto is formed so as to have a shape that can be fitted to a projection part of the subject; and
a processing apparatus configured to process a reflected-wave signal of an ultrasound wave that is transmitted from the ultrasound probe attached to the subject toward the subject.

2. The ultrasound diagnosis apparatus according to claim 1, wherein the ultrasound probe includes:
a plate-like exterior case having the contact face;
a lens of which a curved portion is disposed on the exterior case, the curved portion being formed so as to have a convex shape that can be fitted to a space between bones, each of which is the projection part of the subject; and
a piezoelectric element configured to generate the ultrasound wave transmitted in a substantially thickness direction of the exterior case via the lens.

3. The ultrasound diagnosis apparatus according to claim 1, wherein the ultrasound probe includes:
an exterior case having the contact face that has formed therein a concave portion to be engaged with a bone, which is the projection part of the subject;
a lens provided on the exterior case; and
a piezoelectric element configured to generate the ultrasound wave transmitted in a substantially thickness direction of the exterior case via the lens.

4. The ultrasound diagnosis apparatus according to claim 1, wherein the ultrasound probe is configured so that a transmission angle of the ultrasound wave is changeable by disposing, on the contact face, a member that expands and contracts or a plurality of plate-like adhesive members among which one or more are piled up.

5. The ultrasound diagnosis apparatus according to claim 1, wherein the processing apparatus includes: a controlling unit configured to control the ultrasound probe so as to perform a process of transmitting the ultrasound wave and a process of receiving the reflected-wave signal at one or more points in time that are set in advance.

6. The ultrasound diagnosis apparatus according to claim 1 further comprising: a pulse measuring apparatus that is carried by the subject and is configured to measure a pulse wave or a pulse rate of the subject, wherein
the processing apparatus further includes:
a detecting unit configured to detect whether the subject has an abnormality or not based on the pulse wave or the pulse rate measured by the pulse measuring apparatus; and
a controlling unit configured to, when the detecting unit has detected the abnormality, control the ultrasound probe so as to start a process of transmitting the ultrasound wave and a process of receiving the reflected-wave signal and control the ultrasound probe so as to keep performing the transmitting process and the receiving process until a predetermined period of time has elapsed since the abnormality is detected by the detecting unit.

7. The ultrasound diagnosis apparatus according to claim 6, further comprising: an electrocardiography device configured to obtain an electrocardiogram of the subject, wherein
the processing apparatus further includes: a judging unit configured to judge whether the subject is at a halt by analyzing ultrasound images generated at times when a predetermined waveform is detected in the electrocardiogram, and
the controlling unit controls the ultrasound probe so as to perform the process of transmitting the ultrasound wave and the process of receiving the reflected-wave signal when, as a result of the judging by the judging unit, the subject has transitioned from a moving state into a halt state.
